(19) United States
(12) Patent Application Publication
(10) Pub. No.: US 2014/0127827 A1
(43) Pub. Date: May 8, 2014

(54) METHOD FOR DETECTION OF ANTIGEN USING FLUORESCENCE RESONANCE ENERGY TRANSFER IMMUNOASSAY

(71) Applicant: GWANGJU INSTITUTE OF SCIENCE AND TECHNOLOGY, Gwangju (KR)

(72) Inventors: Min-Gon Kim, Gwangju (KR); Taihua LI, Gwangju (KR); Ju Young BYUN, Gwangju (KR)

(73) Assignee: GWANGJU INSTITUTE OF SCIENCE AND TECHNOLOGY, Gwangju (KR)

(21) Appl. No.: 14/072,079

(22) Filed: Nov. 5, 2013

Related U.S. Application Data
(60) Provisional application No. 61/722,753, filed on Nov. 5, 2012.

(30) Foreign Application Priority Data
Feb. 18, 2013 (KR) 10-2013-0017208

(51) Int. Cl. G01N 33/569

(52) U.S. Cl. CPC G01N 33/56961
USPC 436/501

(57) ABSTRACT

Disclosed herein is a method for detecting an antigen. The method includes: reacting an antigen as an analyte having an absorption wavelength at 300 nm to 400 nm with an antibody to form an antigen-antibody conjugate; irradiating light to the antigen-antibody conjugate to induce fluorescence resonance energy transfer, thereby obtaining a fluorescence spectrum; and determining the presence of the antigen as the analyte by analyzing the fluorescence spectrum, and then measuring the concentration of the antigen. The method is capable of directly detecting various antigens in a homogeneous liquid state, which induces no competitive reaction, with high sensitivity and high selectivity through fluorescent resonance energy transfer, without any modification.

### CROSS-REFERENCE TO RELATED APPLICATION

This application claims priority to Provisional Application Ser. No. 61/722,753 filed on 5 Nov., 2012, and Korean Patent Application No. 10-2013-0017208 filed on 18 Feb., 2013, and all the benefits accruing there from under 35 U.S.C. §119, the contents of which is incorporated by reference in its entirety.

### BACKGROUND

1. Technical Field

The present invention relates to a method for detection of an antigen by binding specific antigens to respective antibodies which specifically bind to the antigens to induce fluorescence resonance energy transfer, thereby directly excluding a competitive reaction without any modification.

2. Description of the Related Art

As a conventional method for detecting an antigen frequently employed in the art, there is an enzyme-linked immunosorbent assay (ELISA), which has been developed to provide good sensitivity, rapid results, convenience and economic feasibility using an immune technique of antigen/antigen, and gradually broadened its application in the art. In a general indirect enzyme-linked immunosorbent assay, it is identified whether a cell line produces a desired antibody by coating an antigen substance (a substance to be analyzed) on a bottom of a plate, adding a cell culture supernatant to allow an antibody present in the supernatant to bind to an antigen, and then allowing color development using another antibody-enzyme conjugate recognizing the antibody bound to the antigen.

However, this method has many drawbacks despite simplicity of testing. First, a direct competitive enzyme-linked immunosorbent assay or an indirect competitive enzyme-linked immunosorbent assay may be used to quantitatively analyze an analyte (a substance to be detected). In the direct competitive enzyme-linked immunosorbent assay, a cell line producing antibodies capable of causing a competitive reaction between an analyte and an analyte-enzyme conjugate must be selected. In the indirect competitive enzyme-linked immunosorbent assay, it is necessary to choice a fusion cell line producing antibodies capable of inducing a competitive reaction between an analyte and an analyte-enzyme conjugate to which a monoclonal antibody is coated. However, for indirect competitive enzyme-linked immunosorbent assay, finally produced antibodies cannot be guaranteed to have a competitive ability to the analyte due to the absence of a stage for to identifying the competitive ability of antibodies. Second, it is said that the indirect enzyme-linked immunosorbent assay is appropriate for the case where the analyte is a large substance such as a protein. This is because a large molecular weight substance is easily coated onto the plate in enzyme-linked immunosorbent assay. Since a low molecular weight substance (for example, mycotoxins such as aflatoxin, ochratoxin, zearalenone and the like; biomarkers such as neopterin and biopterin; polycyclic aromatic carbons; drugs and the like) is not easily coated onto the plate, the low molecular weight substance is not suitable for indirect enzyme-linked immunosorbent assay. Accordingly, when the analyte is a low molecular weight substance, in order for the analyte to be utilized in indirect enzyme-linked immunosorbent assay, a protein having a high molecular weight, i.e., a substance capable of being coated, is bound as a carrier to the analyte, which in turn is coated to the plate, instead of directly coating the analyte to the plate. Subsequently, the addition of a supernatant of a cell culture to the analyte might allow a binding reaction with an antibody recognizing an antigen, but there are antibodies recognizing the carrier or recognizing the binding reaction between the antigen and the carrier, and such antibodies induce color development by secondary antibodies. Examples of methods for detecting antigens using antibodies, besides ELISA, may include lateral flow immunoassays (LFA), electrochemical immunoassays, piezoelectric immunosensors, fluorescence polarization immunoassays, and the like.

Specifically, among low molecular weight antigens to be detected, a mycotoxin is a secondary metabolite produced by fungi, which induces diseases or abnormal physiological function in human and animals, and occurs mostly in grains, nut products and foods where fungi can easily propagate. Accordingly, contamination with mycotoxin in foods is unavoidable, and thus it is essential to examine agricultural products and processed products thereof.

Mycotoxins are largely divided according to genus into aspergillus, penicillium, and fusarium mycotoxins, depending on microorganisms producing the mycotoxins. Further, typical examples of mycotoxin may include aflatoxin, ochratoxin A (OTA) and zearalenone. These mycotoxins are strong pathogenic substances, generally have low solubility in water and are well dissolved in polar organic solvents such as chloroform, methanol, acetonitrile, and the like. Specifically, regardless of surge in interest in the detection of mycotoxins due to sharp increase in global agricultural trade, conventional analysis methods are insufficient for detection of mycotoxins in terms of equipment cost, labor, and the like. In this context, there is a need for a method for effectively analyzing mycotoxins.

In this connection, Korean Patent Publication No. 10-2004-92652A discloses a method for selecting a cell line using a modified direct competitive enzyme-linked immunosorbent assay, a fused cell line producing a monoclonal antibody specific to ochratoxin A obtained there from, a monoclonal antibody and a method for detecting ochratoxin A using such a monoclonal antibody. Korean Patent Publication No. 10-2009-106817A discloses zearalenone-fluorescent tracer for detecting zearalenone by fluorescence-polarization, a method for preparing the same, and a fluorescence-polarization immunoassay of zearalenone using the same.

Further, neopterin among biomarkers, as an analyte, is a low molecular weight substance which is secreted as a pterydine derivative in an activated macrophage upon the stimulation of interferon gamma, and is utilized as a monitor for tracing cell immune system activation. Accordingly, a higher neopterin concentration is indicative of activation of cell immunity. In this regard, it is known that the concentration of neopterin increased in various diseases, including organ transplant rejection mediated by cell immune mechanisms, infectious diseases such as viral infections, autoimmune disorders, tumors and heart failure or renal failure, is indicative of diagnosis and prognosis. Further, it is also known that the concentration of neopterin increases in patients suffering from acute myocardial infarction or unstable angina pectoris. Therefore, rapid and accurate detection of neopterin in blood and urine can play a significant role in early diagnosis and treatment of diseases. Until now, HPLC and enzyme immunoassay have been the most commonly used methods for detection of neopterin.

Polycyclic aromatic carbon (PAH) compounds including benzopyrene are often generated when fossil fuels or organic matter such as plants and the like are incompletely combusted at a temperature from 300° C. to 600° C. Main contaminants include coal tar, automobile exhausted gases (specifically from diesel engines), cigarette smoke, and the like. Benzopyrene may be present in uncooked and unprocessed foods such as agricultural products, fish and shellfish and the like due to environmental pollution. Further, benzopyrene may be generated from carbohydrates, proteins, lipids and the like due to decomposition during processing (IARC, 1987). Many PAHs, including benzopyrene, are toxic and carcinogenic (Group 1), are not readily dissolved in water and remain in the environment for a long period of time in soil deposits, particles in air, thereby causing severe pollution problems. As a method for analyzing PAHs such as benzopyrene, HPLC/FLD (High Performance Liquid Chromatography/Fluorescence Detection) and indirect enzyme immunoassay are generally used.

However, such analyzing methods of prior art have demerits in that the methods require expensive equipment, are time consuming, modification, long time of cultivation, and require several washing stages. Furthermore, these analyzing methods generally indirectly detect a low molecular weight antigen such as a mycotoxin, biomarker, PAH and the like, and thus correspond to immunoassays involving a competitive reaction.

### BRIEF SUMMARY

The present invention has been devised to solve such problems in the art. Specifically, the present invention is directed to providing a method for directly detecting an antigen having an absorption wavelength at 300 nm to 400 nm in a homogeneous liquid state, which induces no competitive reaction, with high sensitivity and high selectivity through fluorescent properties of antibodies in various antigen/antibody conjugates, without any modification.

In accordance with one aspect of the present invention, a method for detecting an antigen includes: reacting an antigen as an analyte having an absorption wavelength at 300 nm to 400 nm with an antibody to form an antigen-antibody conjugate; irradiating light to the antigen-antibody conjugate to induce fluorescence resonance energy transfer, thereby obtaining a fluorescence spectrum; and determining the presence of the antigen as the analyte by analyzing the fluorescence spectrum, and then measuring the concentration of the antigen.

In one embodiment, the antigen may be a mycotoxin, biomarker or polycyclic aromatic carbon having an absorption wavelength near 350 nm.

In another embodiment, the mycotoxin may be aflatoxin, ochratoxin A (OTA), or zearalenone.

In a further embodiment, the biomarker may be neopterin, biopterin, nicotinamide adenine dinucleotide (NADH), or nicotinamide adenine dinucleotide phosphate (NADPH).

In yet another embodiment, the polycyclic aromatic carbon may be benzopyrene, dibenzoanthracene, pyrene, or benzofluorene.

In yet another embodiment, the antigen may be reacted with Fab fragments of the antibody instead of the antibody.

In yet another embodiment, the light causing fluorescence resonance energy transfer may have a wavelength of 260 nm to 290 nm.

In yet another embodiment, analysis of the fluorescence spectrum may be performed by comparing and analyzing the fluorescence intensity at a wavelength showing maximum fluorescence intensity among fluorescence spectra of the antigen or the fluorescence intensity at a wavelength showing maximum fluorescence intensity among fluorescence spectra of the antibody or a Fab fragment of the antibody.

In yet another embodiment, analysis of the fluorescence spectrum may be performed by measuring a ratio [(I_{antigen}/I_{antibody} or I_{antigen}/I_{φαβ antibody}] of fluorescence spectrum intensity (I_{antigen}) at a wavelength showing maximum fluorescence intensity among fluorescence spectra of the antigen to fluorescence intensity (I_{antibody} or I_{φαβ antibody}) at a wavelength showing maximum fluorescence intensity among the fluorescence spectra of the antibody or a Fab fragment of the antibody.

According to the present invention, the method for detection of an antigen is capable of directly detecting various antigens in a homogeneous liquid state, which induces no competitive reaction, with high sensitivity and high selectivity through fluorescent resonance energy transfer, without any modification.

### BRIEF DESCRIPTION OF THE DRAWINGS

The above and other aspects, features, and advantages of the present invention will become apparent from the detailed description of the following embodiments in conjunction with the involving drawings, in which:

FIG. 1a is a schematic view showing the occurrence of FRET between an antibody and an antigen (mycotoxin, biomarker, PAHs, and the like) as an analyte by partial reduction in fluorescence of the antibody due to binding of the antigen to the antibody in a homogeneous liquid state; and FIG. 1b is a schematic view showing the occurrence of FRET between a Fab fragment and an antigen by further reduction in the fluorescence of the Fab fragment due to binding of the antigen to Fab in a homogeneous liquid state;

FIG. 2a is a graph depicting fluorescence spectra of an anti-AFB₁ antibody and Fab fragment excited by light at a wavelength of 280 nm in 250 mM MEST buffer solution (containing 0.3% Tween 20), pH 6.0, and FIG. 2b is a graph depicting a fluorescence excitation wavelength, with fluorescence emitting wavelengths of AFB₁, OTA and ZEN fixed at 450 nm (antibody concentration: 30 μg/mL; Fab fragment concentration: 18 μg/mL; AFB₁ concentration: 500 ng/mL; OTA concentration: 500 ng/mL; ZEN concentration: 1000 ng/mL);

FIG. 3 is a photograph of bands observed in an SDS-PAGE gel for Anti-AFB₁ antibody and Fab fragment of such antibody;

FIGS. 4a to 4c are graphs depicting fluorescence spectra of AFB₁/anti-AFB₁, OTA/anti-OTA and ZEN/anti-ZEN conjugates when 0, 10 and 100 ng/mL of AFB₁ (4a), OTA (4b) and ZEN (4c) form conjugates with antibodies thereof in 25 mM MEST buffer solution (pH 6.0), wherein an excitation wavelength is 280 nm;

FIG. 5 is a schematic view of R₀ (Forster distance) and R (efficient FRET distance) of AFB₁/anti-AFB₁ conjugate and AFB₁/anti-AFB₁ Fab conjugate;

FIG. 6 is a graph depicting fluorescence spectra of AFB₁/anti-AFB₁ conjugate according to various concentrations of AFB₁ in 25 mM MEST buffer solution (pH 6.0), wherein an excitation wavelength is 280 nm;

FIG. 7 is a graph depicting fluorescence spectra of AFB₁/anti-AFB₁ Fab conjugate according to various concentrations of AFB₁ in 25 mM MEST buffer solution (pH 6.0), wherein an excitation wavelength is 280 nm;

FIG. 8 is a graph depicting reduction efficiency of fluorescence intensity at 350 nm of AFB₁/anti-AFB₁ conjugate and AFB₁/anti-AFB₁ Fab conjugate observed from FIGS. 6 and 7, wherein an excitation wavelength is 280 nm;

FIGS. 9a to 9f are fluorescence spectra depicting characteristic features of FRET immunoassay for detecting AFB₁ in 25 mM MEST buffer solution (pH 6.0), wherein an excitation wavelength is 280 nm;

FIG. 10 is a bar graph depicting reduction efficiency of fluorescence intensity at 350 nm in investigation of characteristic features of FRET immunoassay for detecting AFB₁ in FIGS. 9a to 9f, wherein an excitation wavelength is 280 nm;

FIGS. 11a to 11c are graphs showing results of AFB₁ detection in barley spiked with AFB₁ through immunoassay using 25 mM MEST anti-AFB₁ Fab, pH 6.0, wherein an excitation wavelength is 280 nm;

FIG. 12 is a bar graph depicting reduction efficiency of fluorescence intensity at 350 nm as shown in FIGS. 11a to 11c, wherein an excitation wavelength is 280 nm;

FIG. 13 shows a graph (solid line) depicting fluorescence excitation wavelengths of 200 ng/mL neopterin (NPT) by fixing the fluorescence emitting wavelength at 450 nm in 10 mM NaHCO3 buffer solution (pH 9.0) and a graph (dotted line) depicting fluorescence spectra of 200 ng/mL neopterin (NPT) by fixing the fluorescence emitting wavelength at 450 nm in 10 mM NaHCO₃ buffer solution (pH 9.0);

FIG. 14 is a graph depicting fluorescence spectra of NPT/anti-NPT conjugate according to various concentrations of NPT in 10 mM NaHCO₃ buffer solution (pH 9.0), wherein an excitation wavelength is 280 nm;

FIGS. 15a to 15e are fluorescence spectra depicting characteristic features of FRET immunoassay for detecting NPT in 10 mM NaHCO₃ buffer solution (pH 9.0), wherein an excitation wavelength is 280 nm;

FIG. 16 is bar graph depicting reduction efficiency of fluorescence intensity at 350 nm in investigation of characteristic features of FRET immunoassay for detecting NPT in 10 mM NaHCO₃ buffer solution (pH 9.0) as shown in FIGS. 15a to 15e, wherein an excitation wavelength is 280 nm;

FIG. 17 shows a graph (solid line) depicting fluorescence excitation wavelengths of 200 ng/mL benzopyrene (BaP) by fixing the fluorescence emitting wavelength at 435 nm in 10 mM NaHCO₃ buffer solution (pH 9.0) and a graph (dotted line) depicting fluorescence spectra of 200 ng/mL benzopyrene (BaP) excited using light at a wavelength of 380 nm;

FIG. 18 is a graph depicting fluorescence spectra of BaP/anti-BaP conjugate according to various concentrations of BaP in 10 mM NaHCO₃ buffer solution (pH 9.0), wherein an excitation wavelength is 280 nm;

FIG. 19 is a bar graph depicting fluorescence intensity at 435 nm of BaP and BaP/anti-BaP conjugate according to various concentrations of BaP in 10 mM NaHCO₃ buffer solution (pH 9.0), wherein an excitation wavelength is 280 nm;

FIGS. 20a and 20d are fluorescence spectra depicting characteristic features of FRET immunoassay for detecting BaP, wherein an excitation wavelength is 280 nm; and

FIG. 21 is a bar graph depicting a ratio of fluorescence intensity at 435 nm and fluorescence intensity at 350 nm in investigation of characteristic features of FRET immunoassay for detecting BaP as shown in FIGS. 20a to 20d, wherein an excitation wavelength is 280 nm.

### DETAILED DESCRIPTION

Hereinafter, embodiments of the invention will be described in detail with reference to the involving drawings.

Fluorescence resonance energy transfer (FRET) refers to a mechanism describing energy transfer between two adjacent fluorescent substances having two different wavelength regions such that the energy of one fluorescent substance (donor) is transferred to the other fluorescent substance (acceptor or quencher), causing a resonance phenomenon. FRET is a physical phenomenon of energy transfer from one excited dye molecule to the other dye molecule by long distance dipole-dipole interaction where a molecule donating the energy is called donor and a molecule accepting the energy is called acceptor or quencher.

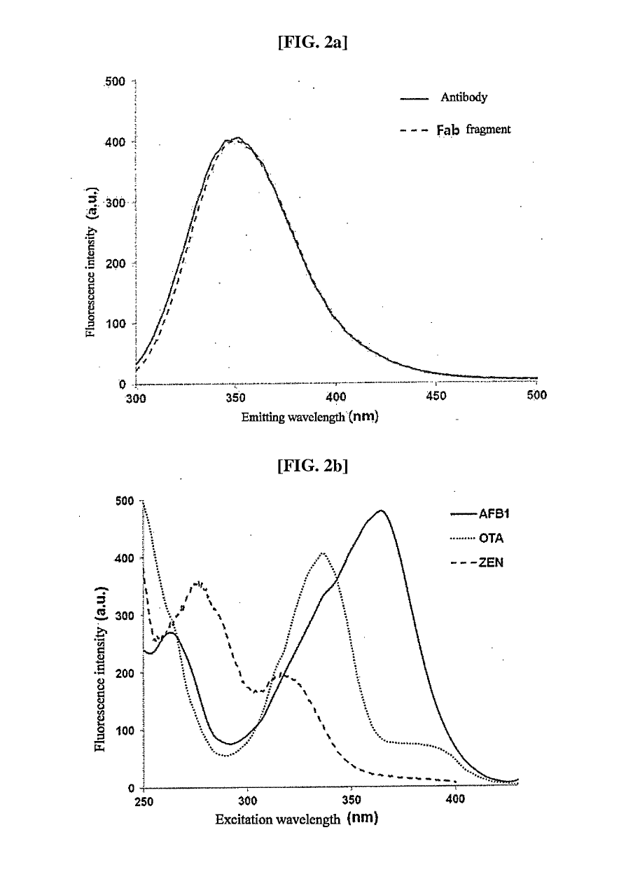

When a donor molecule is excited by light of a specific wavelength, and if an acceptor molecule is in close proximity with the donor molecule, the energy of the donor molecule is transferred to the acceptor molecule, causing reduction in fluorescence of the donor molecule while causing fluorescence of the acceptor molecule, or causing only reduction in fluorescence of the donor molecule. Such an energy transfer phenomenon may occur when the donor and the acceptor are separated by a distance of around 1˜10 nm. The fundamental condition for such energy transfer is that the fluorescence spectrum of the donor molecule overlaps the absorption spectrum of the acceptor molecule. Proteins such as antibodies have a maximum absorption wavelength at 260 nm˜290 nm, approximately at 280 nm due to the presence of tryptophan (Trp). When light in such wavelength bands is irradiated to proteins, the proteins exhibit the highest fluorescence signal at 300 nm˜400 nm, approximately at 350 nm (see FIG. 2a and FIG. 2b). Accordingly, as described above, it is noted that the absorption wavelength of fluorescent antigens, for example, mycotoxins such as aflatoxin, ochratoxin, zearalenone, and the like, overlaps the fluorescence spectrum of proteins, such as antibodies. By utilizing such phenomenon, a method for detecting fluorescent antigens can be provided.

Namely, the present inventors have developed a method for directly detecting an antigen without modification by excluding a competitive reaction by reacting antigens with antibodies specific to the antigens to induce fluorescence resonance energy transfer. Therefore, the method for detection of an antigen according to the present invention includes reacting an antigen as an analyte having an absorption wavelength at 300 nm to 400 nm with an antibody to form an antigen-antibody conjugate; irradiating light to the antigen-antibody conjugate to induce fluorescence resonance energy transfer, thereby obtaining a fluorescence spectrum; and determining the presence of the antigen as the analyte by analyzing the fluorescence spectrum, and then measuring the concentration of the antigen.

As the antigen detectable by the method according to the invention, any antigen capable of inducing FRET may be used without limitation. Examples of the antigen may include mycotoxins, biomarkers or polycyclic aromatic carbons, which have a wavelength around 350 nm, without being limited thereto. Specifically, examples of the mycotoxin may include aflatoxin, ochratoxin A (OTA), or zearalenone. Examples of the biomarker may include neopterin, biopterin, nicotinamide adenine dinucleotide (NADH), or nicotinamide adenine dinucleotide phosphate (NADPH). Examples of the polycyclic aromatic carbon may include benzopyrene, dibenzoanthracene, pyrene, or benzofluorene.

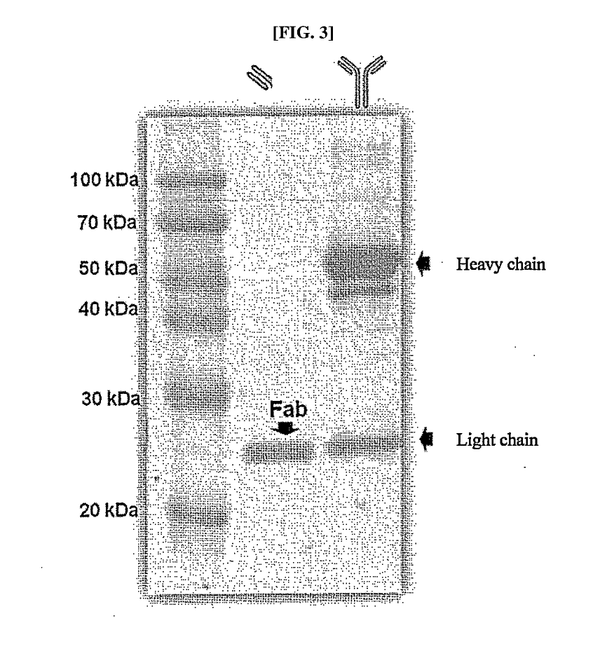

As substances causing an antigen/antibody reaction with a mycotoxin, an antibody against the mycotoxin may be used, as described above. Instead of the antibody, Fab fragments of the antibody may also be used. Here, Fab fragments refer to portions corresponding to light chains of an antibody among the intact antibody including heavy chains and light chains (see FIG. 3). As can be seen from results of examples, when using Fab fragments instead of an intact antibody, sensitivity of immunoassay will be further enhanced and thus a mycotoxin can be detected with a sensitivity 10 times or more better than in the case where an intact antibody is used.

As mentioned above, after an antigen as an analyte is reacted with an antibody against the antigen or with the Fab fragment of the antibody, the formed antigen/antibody conjugate is irradiated with light to induce fluorescence resonance energy transfer, thereby obtaining a fluorescence spectrum. Subsequently, the fluorescence spectrum is analyzed to determine the presence of the antigen as the analyte, and the concentration of the antigen is measured.

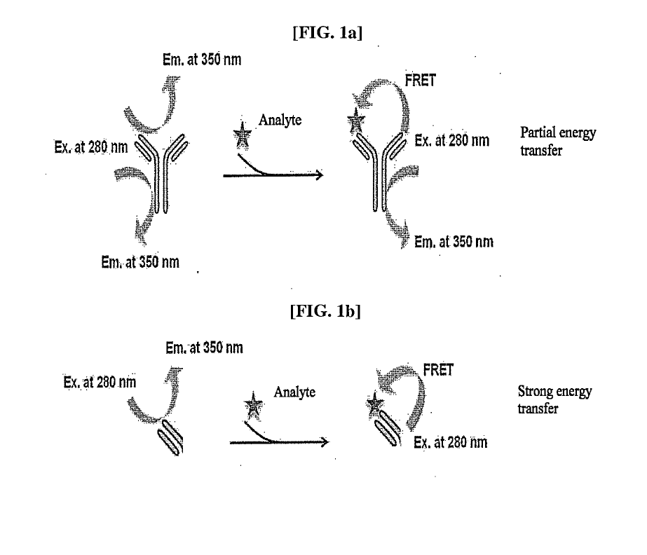

Specifically, a tryptophan residue of an antibody protein exhibits a maximum absorption at 280 nm and a maximum emission at 350 nm Therefore, analysis of the fluorescence spectrum may be performed through excitation using light having a wavelength of 280 nm and observing an emitting spectrum at 350 nm Namely, referring to FIGS. 1a and 1b, when a mycotoxin is present in a sample, an antibody binds to the mycotoxin, causing a quenching phenomenon. The more mycotoxin is present in a sample, the higher the degree of quenching becomes, and thus, reduction in fluorescence intensity at 350 nm becomes more remarkable. On the contrary, when a mycotoxin is not present in a sample, such a quenching phenomenon does not occur, and thus, the fluorescence intensity at 350 nm becomes stronger.

In this regard, analysis of the fluorescence spectrum may be performed by analyzing the fluorescence intensity at a wavelength showing maximum fluorescence intensity among fluorescence spectra of the antigen or the fluorescence intensity at a wavelength showing maximum fluorescence intensity among fluorescence spectra of the Fab fragment of the antibody.

Alternatively, analysis of the fluorescence spectrum may be performed by measuring a ratio [(I_{antigen}/I_{antibody} or I_{antigen}/I_{φαβ antibody}] of fluorescence intensity (I_{antigen}) at a wavelength showing maximum fluorescence intensity among the fluorescence spectra of the antigen to fluorescence intensity (I_{antibody} or I_{φαβ antibody}) at a wavelength showing maximum fluorescence intensity among the fluorescence spectra of the antibody or Fab fragment of the antibody.

As set forth above, use of only the Fab fragment of the antibody against the mycotoxin (FIG. 1b) exhibits more remarkable reduction as compared to the use of the intact antibody against the mycotoxin (FIG. 1a), which further improves sensitivity of immunoassay.

Next, the present invention will be better appreciated from the following examples. It should be understood that these examples are provided for illustration only and are not to be construed in any way as limiting the scope of the present invention.

### Example 1

### Detection of Mycotoxin Through FRET Immunoassay Using an Intact Antibody

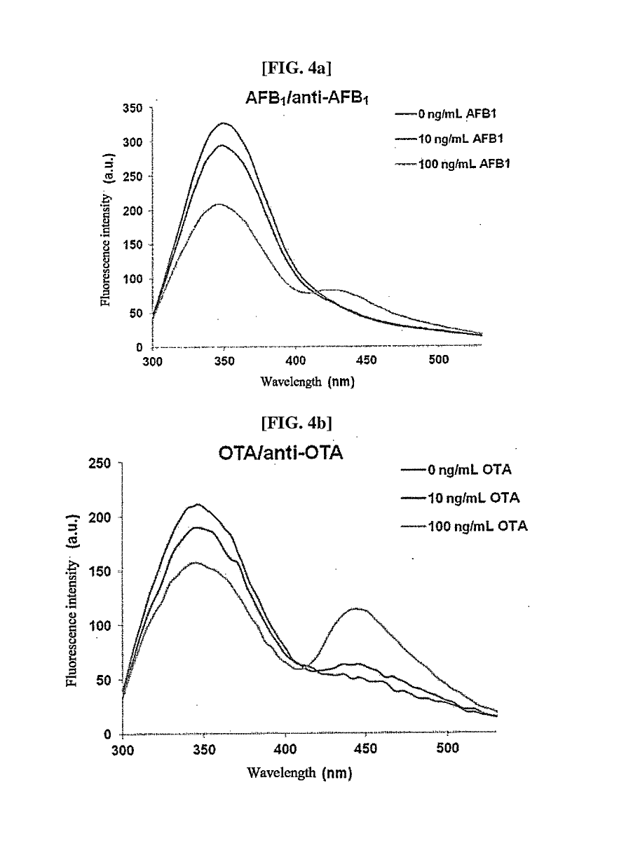

Using 25 mM MEST buffer solution (containing 0.3% Tween 20 in MES buffer solution, pH 6.0), mycotoxins of various concentrations (0 ng/mL, 10 ng/mL and 100 ng/mL) were reacted with antibodies having a constant concentration at room temperature for 20 minutes to obtain mycotoxin/antibody conjugates (AFB₁/anti-AFB₁ conjugate, OTA/anti-OTA conjugate, and ZEN/anti-ZEN conjugate). The obtained mycotoxin/antibody conjugates were excited at 280 nm to obtain fluorescence spectra (FIG. 4). Referring to FIG. 4, it can be seen that, as the concentration of mycotoxins (AFB₁, OTA and ZEN) increases, the fluorescence intensity at a wavelength of 350 nm decreases.

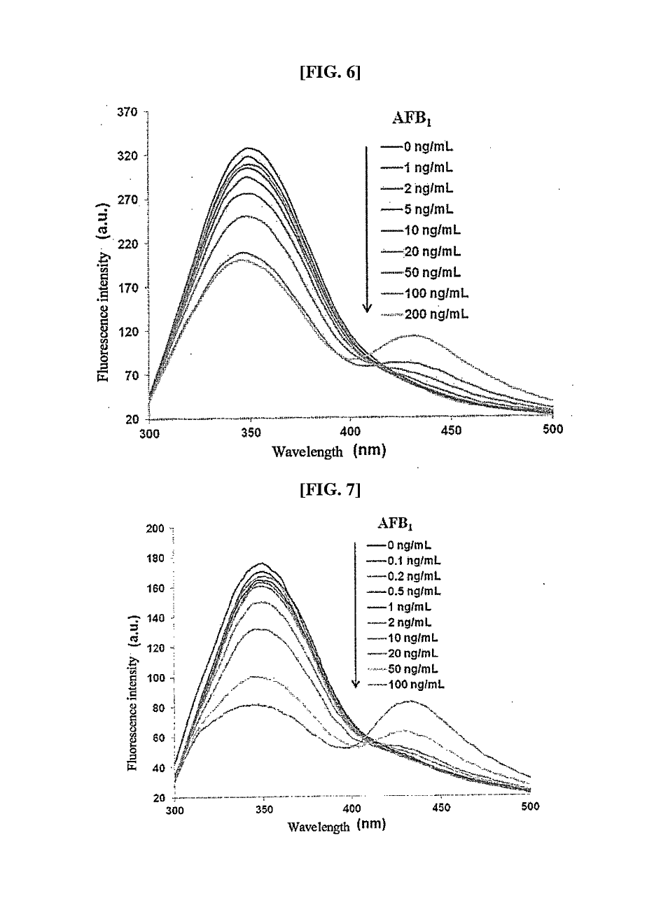

Specifically, in order to analyze reduction in fluorescence intensity depending on the concentration of mycotoxins in detail, reduction in fluorescence intensity was analyzed using AFB₁ at concentrations of 0, 1, 2, 5, 10, 20, 50 and 100 (ng/mL), and results are shown in FIG. 6. Referring to FIG. 6, it can be seen that, as the concentration of mycotoxins increases, reduction in fluorescence intensity at 350 nm becomes more remarkable. Further, as a result of analysis by lowering the concentration of mycotoxins, it can be seen that approximately up to 0.85 ng/mL of the mycotoxins can be detected.

### Example 2

### Detection of Mycotoxin Through FRET Immunoassay Using Fab Fragment (Anti-AFB₁ Fab)

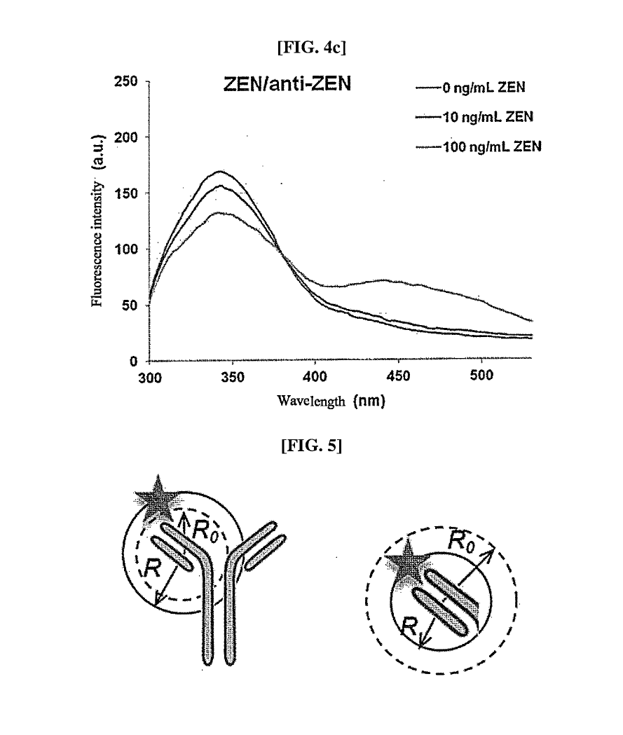

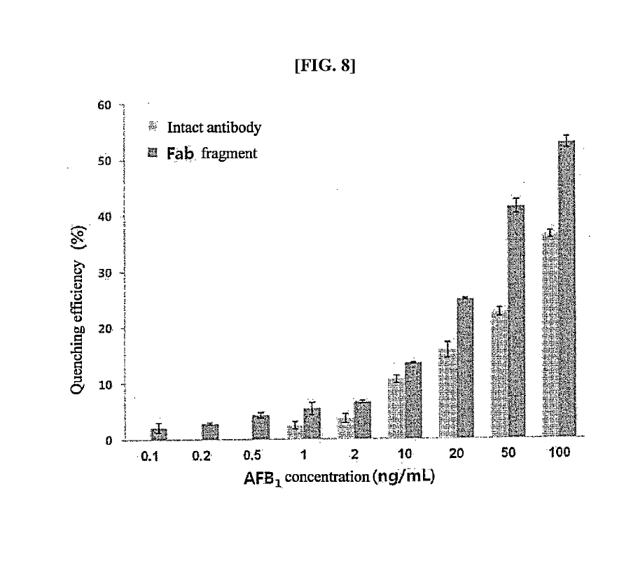

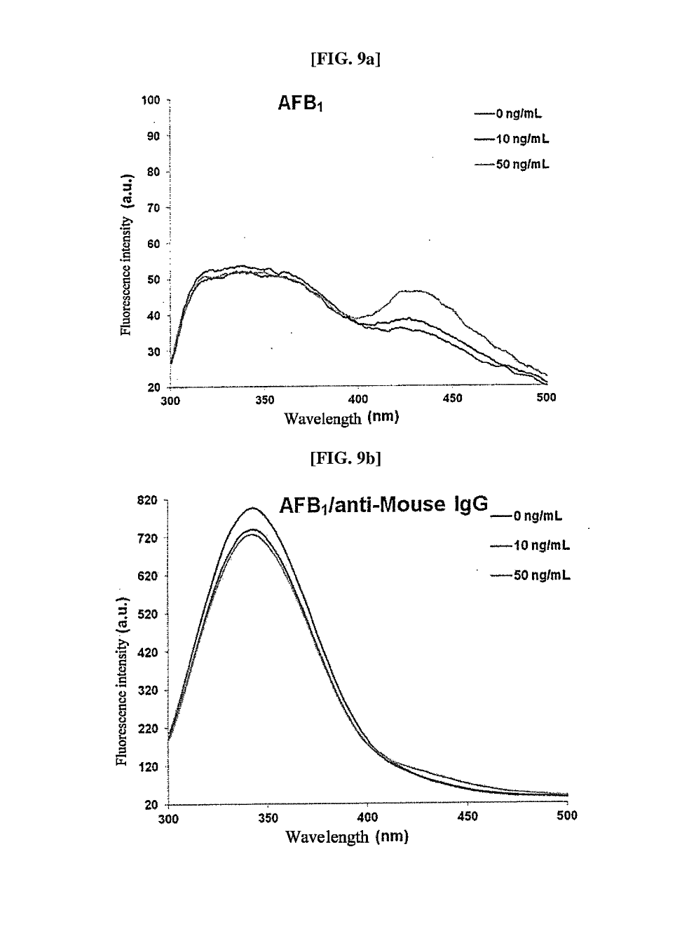

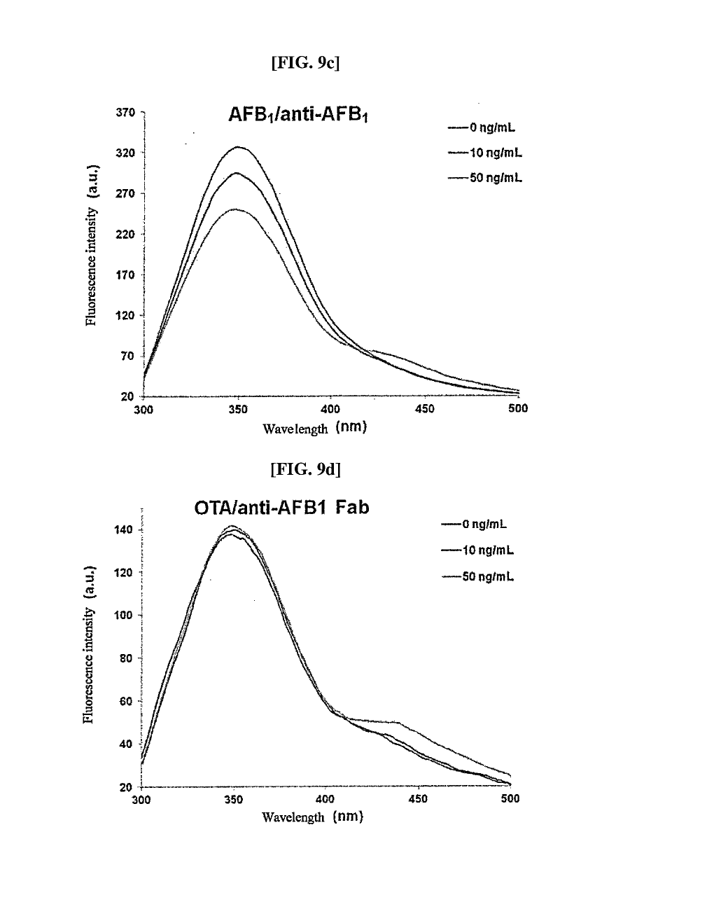

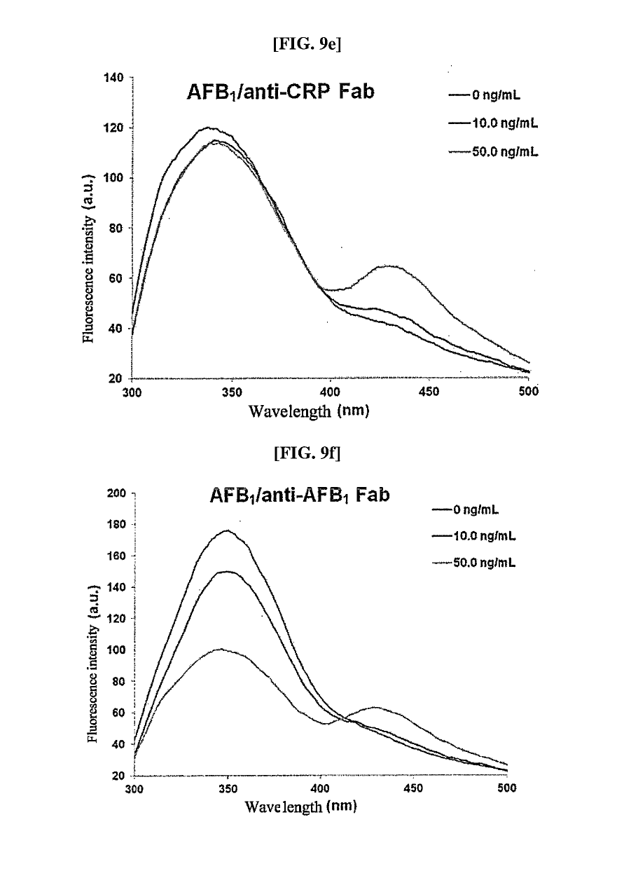

Under the condition of 25 mM MEST buffer solution (pH 6.0), AFB₁ of various concentrations (0, 0.1, 0.2, 0.5, 1, 2, 10, 20, 50 and 100 ng/mL) was reacted with anti-AFB₁ Fab fragments having a constant concentration at room temperature for 20 minutes to obtain AFB₁/anti-AFB₁ Fab conjugates. The obtained AFB₁/anti-AFB₁ Fab conjugates were excited at 280 nm to obtain fluorescence spectra (FIG. 7). Referring to FIG. 7, it can be seen that, as the concentration of AFB₁ increases, the fluorescence intensity at a wavelength of 350 nm decreases. Particularly, it can also be seen that sensitivity of FRET immunoassay using Fab fragments was much better than that of FRET immunoassay using an intact antibody of Example 1 (FIG. 8). Further, as a result of analysis by lowering the concentration of AFB₁, it can be seen that approximately up to 0.09 ng/mL of AFB₁ could be detected through FRET immunoassay using Fab fragments. This corresponds to a near 10-fold improvement in terms of sensitivity as compared to FRET immunoassay where an intact antibody of Example 1 was used. As depicted in FIG. 5, it is estimated that the results were caused by the fact that the efficient FRET distance (indicated as R in FIG. 5) in an AFB₁/anti-AFB₁ conjugate increases as compared with Forester distance (indicated as R₀ in FIG. 5), whereas the efficient FRET distance in AFB₁/anti-AFB₁Fab conjugate becomes closer as compared with Forester distance.

### Example 3

### Specificity Investigation of AFB₁ Detection Using FRET Immunoassay

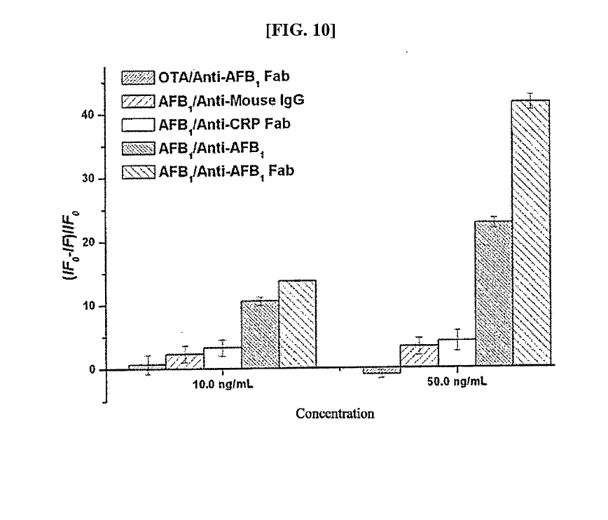

Under the condition of MEST buffer solution (pH 6.0), specificity of mouse IgG antibody (anti-Mouse IgG) and CRP antibody Fab fragment (anti-CRP Fab) to 10 ng/mL and 50 ng/mL of AFB₁ was investigated using FRET immunoassay. As a result, it can be seen that the two antibodies did not substantially bind to AFB₁. Further, specificity of anti-AFB₁ Fab to OTA having the same concentration as AFB₁ was investigated. As a result, it can be seen that there was no antigen/antibody binding and no specificity was observed (see FIGS. 10 and 10). From these results, it can be seen that FRET immunoassay using the AFB₁/anti-AFB₁ conjugate exhibited strong specificity only between AFB₁ and anti-AFB₁ or anti-AFB₁ Fab.

### Example 4

### Detection of Mycotoxin in Barley Spiked with Mycotoxin Using FRET Immunoassay

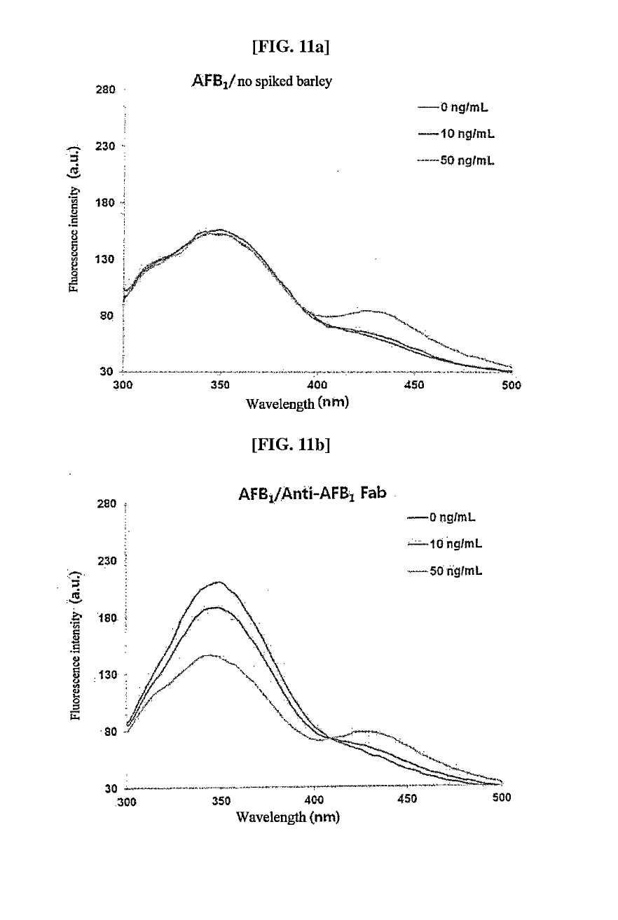

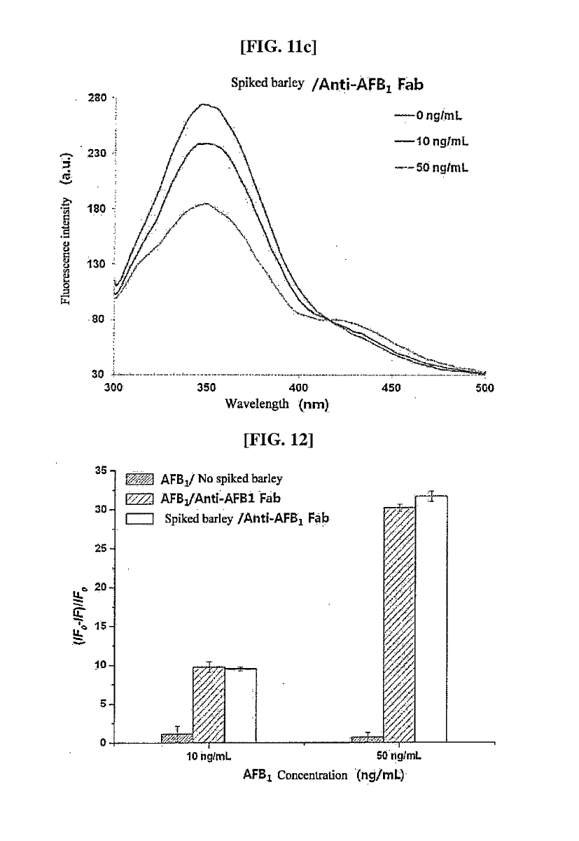

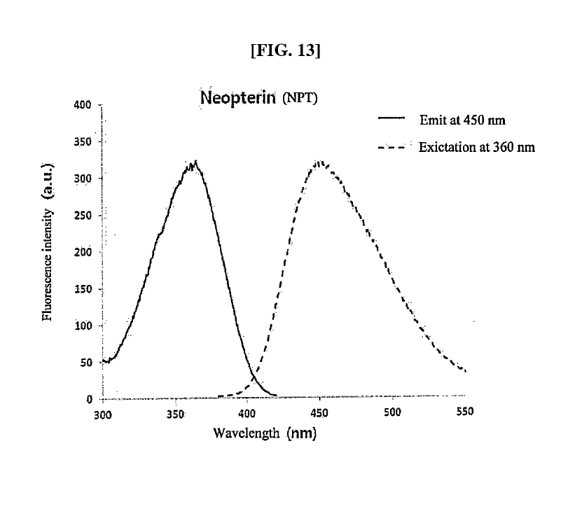

After barley was spiked with 10 and 50 ng/mL of AFB₁ (60% methanol solution), AFB₁ was extracted using a 0.45 nm membrane filter. Subsequently, an experiment detecting AFB₁ was performed by FRET immunoassay using Fab fragments. As a result, it was observed that at the same concentration of AFB₁, reduction efficiency in fluorescence intensity of AFB₁/anti-AFB1 Fab at 350 nm was very similar to the reduction efficiency in the fluorescence intensity of AFB₁/anti-AFB1 Fab at 350 nm in barley sample spiked with AFB₁ (see FIGS. 11 and 12). From this fact, it can be seen that AFB₁ can be directly detected in real samples.

### Example 5

### Detection of Biomarker (Neopterin, NPT) Through FRET Immunoassay Using Antibodies

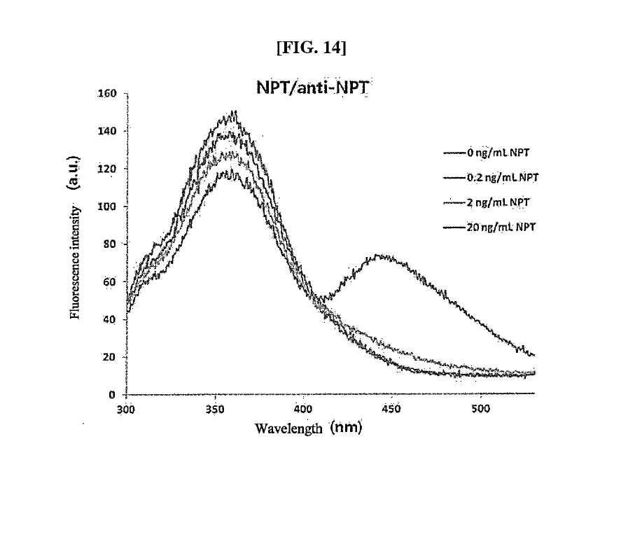

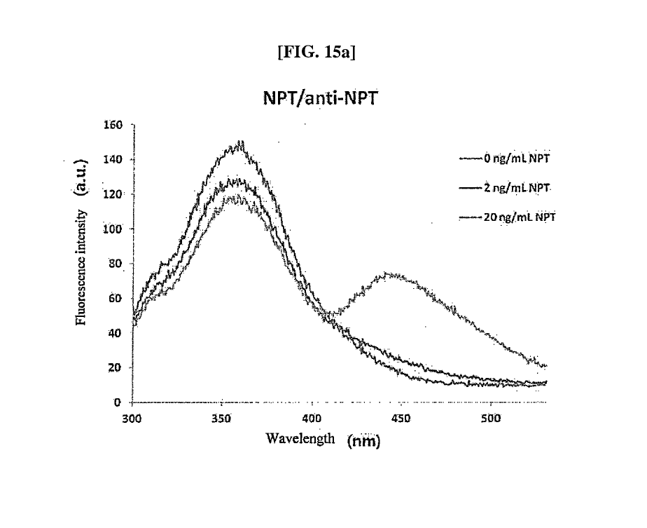

Under the condition of 10 mM NaHCO₃ buffer solution (pH 9.0), NPT of various concentrations (0 ng/mL, 0.2 ng/mL, 2 ng/mL and 20 ng/mL) was reacted with antibodies having a constant concentration at room temperature for 20 minutes to obtain NPT/anti-NPT conjugates. The obtained NPT/anti-NPT conjugates were excited at 280 nm to obtain fluorescence spectra (FIG. 14). Referring to FIG. 14, it can be seen that, as the concentration of NPT increases, the fluorescence intensity at a wavelength of 350 nm decreases.

### Example 6

### Specificity Investigation for NPT Detection Using FRET Immunoassay

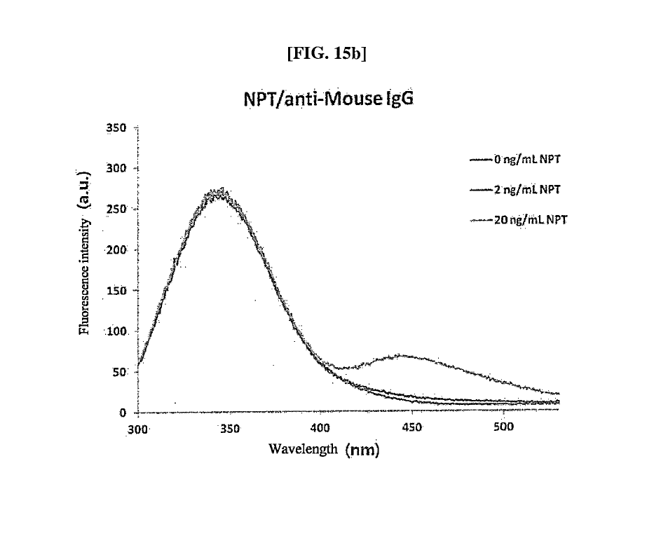

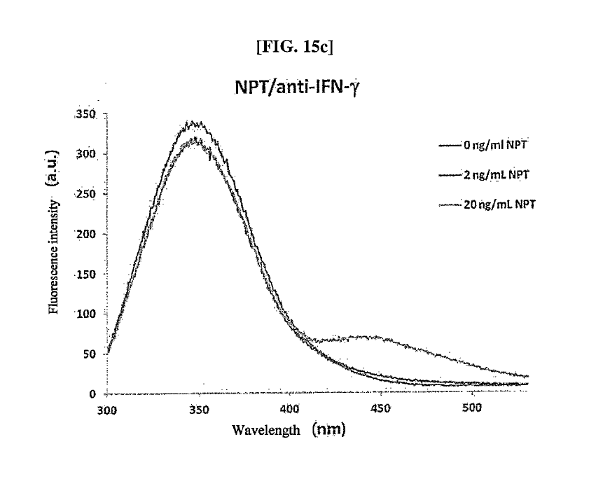

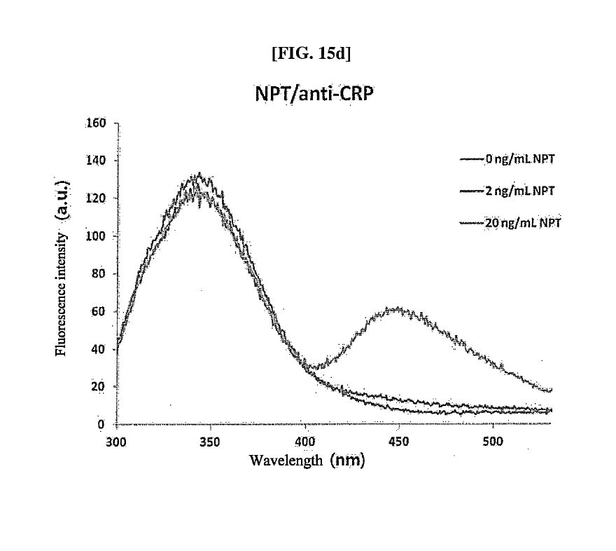

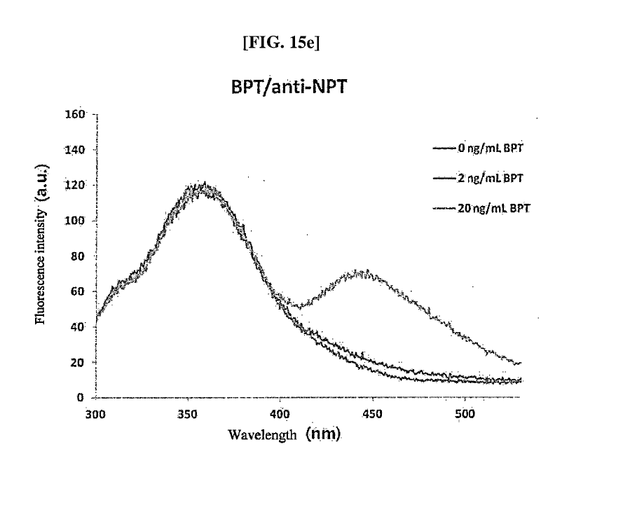

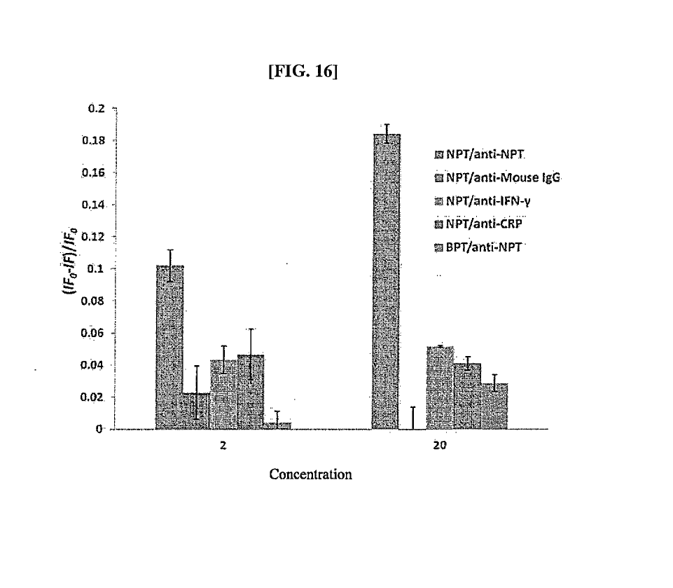

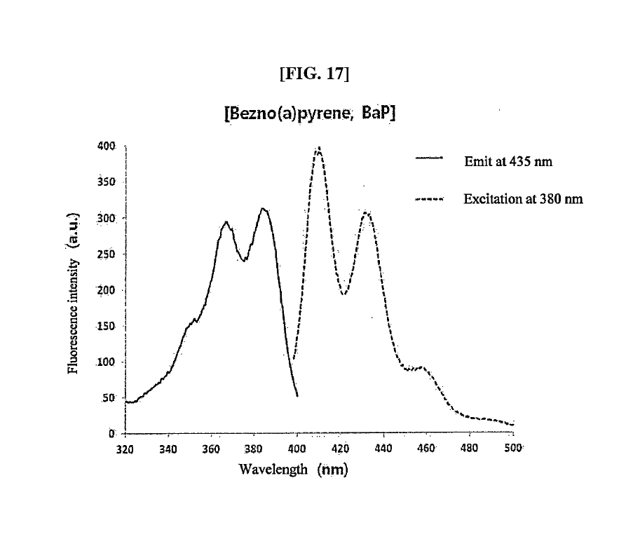

Under the condition of 10 mM NaHCO₃ buffer solution (pH 9.0), specificity of mouse IgG antibody (anti-Mouse IgG), IFN-γ antibody (anti-IFN-γ) and CRP antibody (anti-CRP) to 2 ng/mL and 20 ng/mL of NPT was investigated using FRET immunoassay. As a result, it can be seen that these antibodies did not substantially bind to NPT. Further, specificity of anti-NPT with BPT having the same concentration as NPT was investigated. As a result, it was seen that there was no antigen/antibody binding and no specificity was observed (see FIGS. 15 and 16). From these experimental results, it can be seen that FRET immunoassay using NPT/anti-NPT conjugate exhibits strong specificity only between NPT and anti-NPT.

### Example 7

### Detection of PAH (Benzopyrene, BaP) Through FRET Immunoassay Using Antibodies

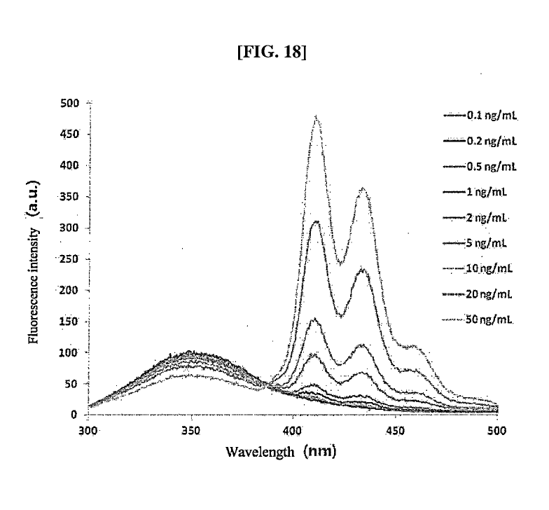

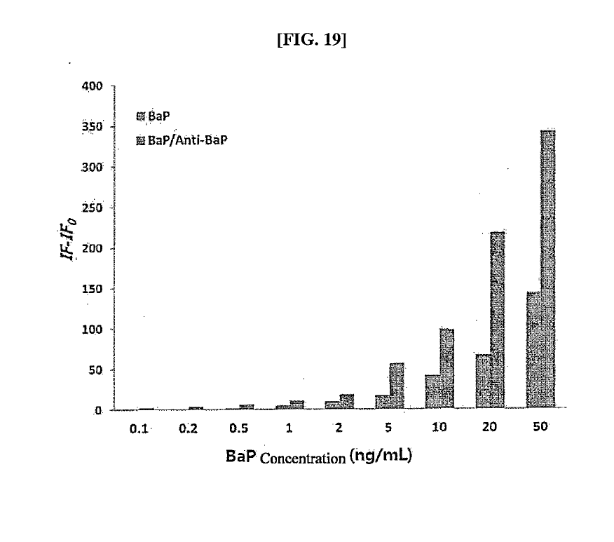

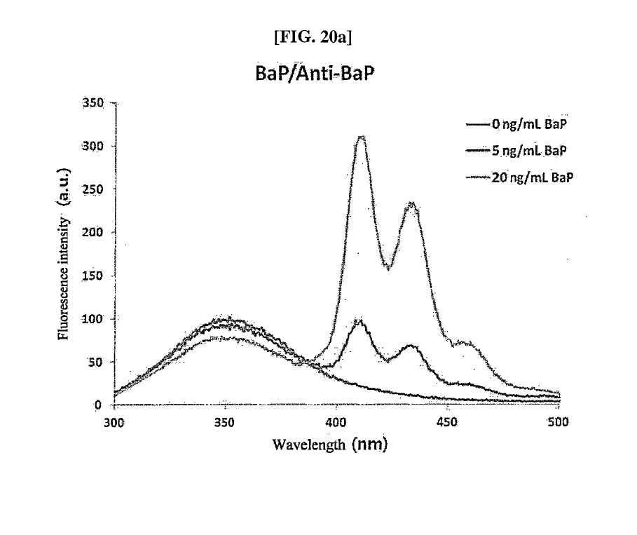

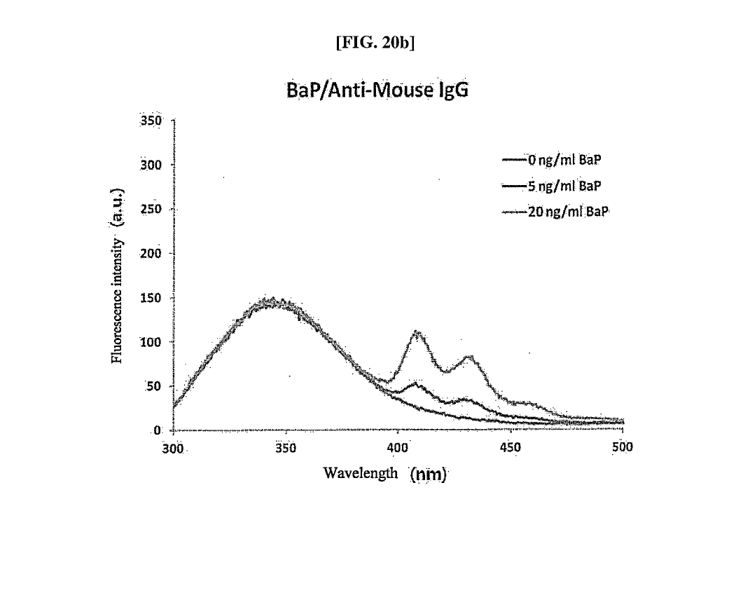

Under the condition of 10 mM NaHCO₃ buffer solution (pH 9.0), BaPs having various concentrations (0, 0.1, 0.2, 0.5, 1, 2, 5, 10, 20 and 50 ng/mL) were reacted with antibodies having constant concentration at room temperature for 20 minutes to obtain BaP/anti-BaP conjugates. The obtained BaP/anti-BaP conjugates were excited at 280 nm to obtain a fluorescence spectrum (FIG. 18). Referring to FIGS. 18 and 19, it can be seen that, as the concentration of BaP increases, fluorescence intensity at a wavelength of 350 nm decreases, while fluorescence intensity at 435 nm increases at the same time.

### Example 8

### Specificity Investigation for BAP Detection Using FRET Immunoassay

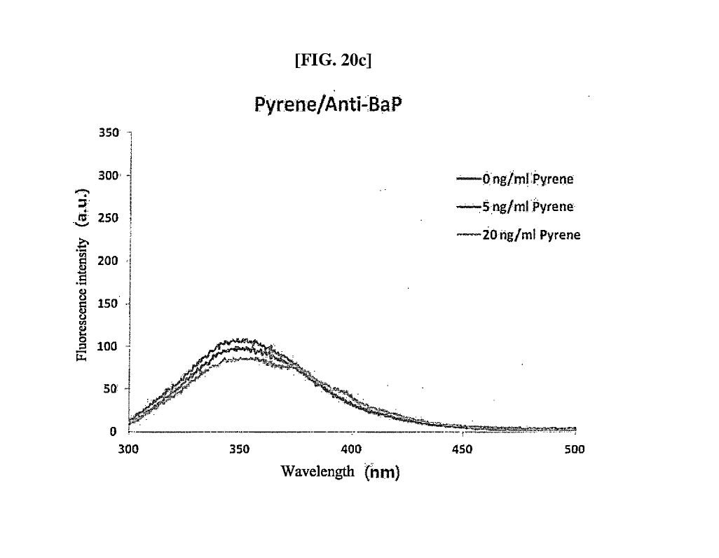

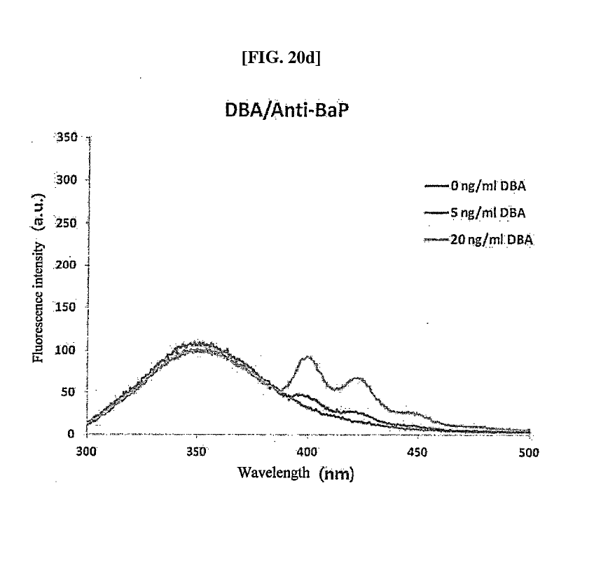

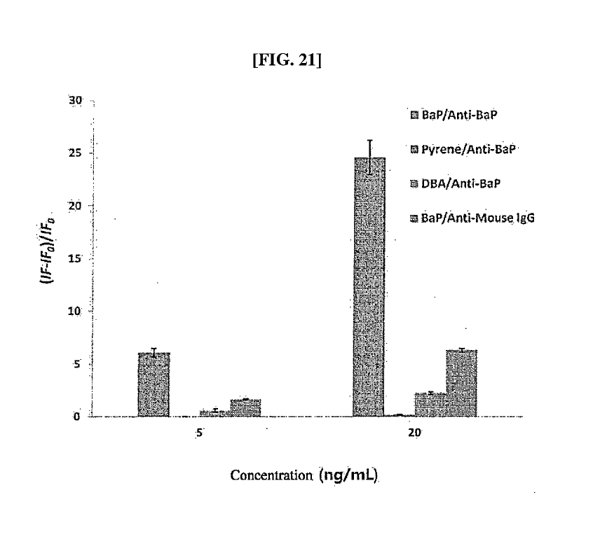

Under the condition of 10 mM NaHCO₃ buffer solution (pH 9.0), specificity of mouse IgG antibody (anti-Mouse IgG) to 5 ng/mL and 20 ng/mL BaP was investigated using FRET immunoassay. As a result, it can be seen that the antibody did not substantially bind to BaP. Further, specificity of anti-BaP to pyrene and dibenz[a,h]anthracene (DBA) having the same concentration as BaP was investigated. As a result, it can be seen that there was a little antigen/antibody binding, but the specificity was much inferior to BaP (FIGS. 20 and 21). From these experimental results, it can be seen that FRET immunoassay using BaP/anti-BaP conjugate exhibits a strong specificity between BaP and anti-BaP.

Although some exemplary embodiments have been described herein, it should be understood by those skilled in the art that these embodiments are given by way of illustration only, and that various modifications, variations and alterations can be made without departing from the spirit and scope of the invention. The scope of the present invention should be defined by the appended claims and equivalents thereof.

What is claimed is:

1. A method for detecting an antigen, comprising:
reacting an antigen as an analyte having an absorption wavelength at 300 nm to 400 nm with an antibody to form an antigen-antibody conjugate;
irradiating light to the antigen-antibody conjugate to induce fluorescence resonance energy transfer, thereby obtaining a fluorescence spectrum; and
determining the presence of the antigen as the analyte by analyzing the fluorescence spectrum, and then measuring the concentration of the antigen.

2. The method according to claim 1, wherein the antigen is a mycotoxin, a biomarker or a polycyclic aromatic carbon, having an absorption wavelength near 350 nm.

3. The method according to claim 2, wherein the mycotoxin is aflatoxin, ochratoxin A (OTA), or zearalenone.

4. The method according to claim 2, wherein the biomarker is neopterin, biopterin, nicotinamide adenine nucleotide (NADH), or nicotinamide adenine nucleotide phosphate (NADPH).

5. The method according to claim 2, wherein the polycyclic aromatic carbon is benzopyrene, dibenzoanthracene, pyrene, or benzofluorene.

6. The method according to claim 1, wherein the antigen is reacted with a Fab fragment of the antibody instead of the antibody.

7. The method according to claim 1, wherein the light causing fluorescence resonance energy transfer has a wavelength of 260 nm to 290 nm.

8. The method according to claim 6, wherein analysis of the fluorescence spectrum is performed by comparing and analyzing the fluorescence intensity at a wavelength showing maximum fluorescence intensity among fluorescence spectra of the antigen or the fluorescence intensity at a wavelength showing maximum fluorescence intensity among the fluorescence spectra of the antibody or a Fab fragment of the antibody.

9. The method according to claim 6, wherein analysis of the fluorescence spectrum is performed by measuring a ratio [(I_{antigen}/I_{antibody} or I_{antigen}/I_{φαβ antibody}] of fluorescence spectrum intensity (I_{antigen}) at a wavelength showing maximum fluorescence intensity among fluorescence spectra of the antigen to fluorescence intensity (I_{antibody} or I_{φαβ antibody}) at a wavelength showing maximum fluorescence intensity among the fluorescence spectra of the antibody or a Fab fragment of the antibody.
